(19) United States
(12) Patent Application Publication
(10) Pub. No.: US 2024/0000756 A1
(43) Pub. Date: Jan. 4, 2024

(54) METHODS AND COMPOUNDS FOR TREATMENT OF AUTISM SPECTRUM DISORDER

(71) Applicant: Crestone, Inc., Boulder, CO (US)

(72) Inventors: Nebojsa Janjic, Boulder, CO (US); Urs Ochsner, Boulder, CO (US)

(73) Assignee: Crestone, Inc., Boulder, CO (US)

(21) Appl. No.: 18/038,401

(22) PCT Filed: Dec. 8, 2021
(86) PCT No.: PCT/US21/62454

§ 371 Date: May 23, 2023

Related U.S. Application Data
(60) Provisional application No. 63/123,453, filed on Dec. 9, 2020.

(51) Int. Cl. A61K 31/4353; A61K 35/747; A61P 31/04; A61P 25/00

(52) U.S. Cl. CPC A61K 31/4353; A61K 35/747; A61P 31/04; A61P 25/00

(57) ABSTRACT

Methionyl tRNA synthetase inhibitors (MetRS) are provided for use in therapy as antibacterial agents in autism spectrum disorder.

### TECHNICAL FIELD

The present invention relates to the use of bacterial methionyl tRNA synthetase (MetRS) inhibitors as antibacterial agents in the treatment of autism spectrum disorders (ASD), and in particular to the use of MetRS inhibitors in the treatment of autism spectrum disorders (ASD) associated with Clostridium infection.

### BACKGROUND OF THE INVENTION

Autism spectrum disorder is an increasingly prevalent neurodevelopmental disorder that currently affects one in 68 children in the U.S., with boys affected 4.5-times more often than girls (Christensen et al., MMWR Surveillance Summ. (2016) 65:1-28). Symptoms, which cover a wide range of severities, include impairments in communication, reduced social interactions, attenuated language development, repetitive behaviors and restricted interests. Prevalence of 1-2% is similar in North America, Europe and Asia, affecting all racial, ethnic and socioeconomic groups. With the exception of rare cases with defined genetic abnormalities, for example, as observed with Fragile X syndrome, neither the cause nor the reason for the increase in prevalence of autism is well understood. There are currently no FDA-approved treatments for autism.

There is increasing evidence for the existence of gut-brain connection in autism (Frye et al. Microbial Ecol. Health Dis. (2016) 26:26878; Ding et al., J. Autism Dev. Disord. (2017) 47:480-89; Li et al. Front. Cell. Neurosci. (2017) 11:120; Vuong et al., Biol. Psychiatry (2017) 81:411-23). Gastrointestinal (GI) symptoms are frequent in autistic kids. Constipation, diarrhea, or alternating episodes of constipation and diarrhea are a common occurrence (Bresnahan et al., JAMA Psychiatry (2015) 72:466-74; McElhanon et al., Pediatrics (2014) 133:872-83). GI symptoms are strongly correlated with severity of autism (Chaidez et al., J. Autism Dev. Disord. (2014) 44:1117-27; Adams et al., BMC Gastroenterol. (2011) 11:22). Altered GI motility and increased intestinal permeability (“leaky gut”) are common (Hsiao et al., Cell (2013) 155:1451-63). A considerable body of evidence now supports the notion that the gut microbiome is different and significantly less diverse in autistic kids, at least in a subset of individuals, compared with neurotypical (normal) kids (Kang et al., PLoS One (2013) 8:e68322; Kang et al., Microbiome (2017) 5:10). See also Sharon et al. ((2019) Cell 177, 1600) and Kang et al. ((2019) Sci. Reports 9, 5821).

In about one-third of the cases, an initial period of normal childhood development is followed by the onset of developmental regression, in what has been described as late-onset or regressive autism (Sandler et al., J. Child Neurol. (2000) 15:429-35). Affected kids have a history of increased antibiotics use for recurring infections (e.g., otitis media) prior to the onset of autism symptoms (Niehus et al., J. Dev. Behav. Pediatr. (2006) 27:S120-7), and onset of symptoms often follows antimicrobial therapy (Finegold, Med. Hypotheses (2008) 70:508-11). Dysbiosis in autism exhibits directional trends characterized with an increase in Clostridum clusters I, II and XI (but not clusters XIVa and b), an increase in Lactobacilli, and a reduction in Bifidobacteria and Bacteroides (Finegold, Med. Hypotheses (2008) 70:508-11; Parracho et al., J. Med. Microbiol. (2005) 54:987-91; Finegold et al., Anaerobe (2017) 45:133-37; Adams et al., BMC Gastroenterol. (2011) 11:22; Hsiao et al., Cell (2013) 155:1451-63).

In this context, Clostridia in particular have received considerable attention. Most gut Clostridia are spore formers that can survive antibiotic treatment, followed by germination and overgrowth of commensal gut flora (Finegold (2008), supra; Parracho et al. (2005), supra; Ding et al., J. Autism Dev. Disord. (2017) 47:480-89; Finegold (2017), supra). In addition, Clostridia are the principal producers of enterotoxins, neurotoxins and potentially toxic metabolites, such as phenols, p-cresol and indole derivatives (Finegold (2008), supra; Parracho et al. (2005), supra; Ding et al. (2017), supra; Finegold (2017), supra). Short generation time of Clostridia allows them to rapidly proliferate in permissive settings, such as in opportunistic conditions typical in dysbiotic gut (Finegold et al. (2017), supra). In a recent study in which gut flora from thirty-three autistic kids with GI abnormalities was compared with that of thirteen neurotypical kids without GI symptoms, autistic kids had more than 10-fold higher stool counts of C. perfringens (2.1×10⁵ CFU/g vs. 1.7×10⁴ CFU/g, respectively), a statistically significant difference (Finegold et al. (2017) supra). C. perfringens beta2, alpha and enterotoxin were also detected in a higher proportion in autistic kids compared with neurotypical kids (Finegold et al. (2017), supra). As a rapid grower and a prolific producer of toxins, C. perfringens is a well-known human and animal pathogen. In humans, C. perfringens is the second most common bacterial cause of food poisoning, which in most cases originates from contaminated meat and poultry (Grass et al., Foodborne Pathog. Dis. (2013) 10:131-136). C. perfringens can also cause toxin-mediated soft tissue necrosis of various severities and, in extreme cases, a life-threatening condition called gas gangrene (Finegold et al. (2017) supra). In livestock, C. perfringens toxemia is an important disease in grazing animals, especially in lambs, goats and calves (Finnie, Anaerobe (2004) 10:145-50). In lambs, toxemia caused by epsilon toxin is well studied and is typically manifested in neurological disorders that can frequently be fatal. The disease, also known as “overeating disease”, is often initiated by heavy consumption of starch-rich crops that favor the overgrowth of saccharolytic bacteria such as C. perfringens. Active intestinal peristalsis attenuates the overgrowth of C. perfringens, but if such gut motility is slowed, toxins can accumulate in localized regions and expand to levels that cause an increase in intestinal permeability which leads to leakage of the toxin into systemic circulation. Once in the bloodstream, toxin selectively accumulates in the brain, and to a lesser degree, the kidneys. Accumulation in the brain, which appears to be driven by specific toxin receptors on the luminal side of brain microvascular endothelium, causes disruption of the blood-brain barrier, severe cerebral edema and neuronal cytotoxicity (Finnie (2004) supra). Thus, in domesticated animals, C. perfringens, which resides in the intestines, is a well-known cause of neurological disorders, caused by Clostridium toxins.

Clostridium bacteria are a spore forming family of Gram-positive anaerobes, including Clostridium(C) perfringens, C. tetani, C. botulinum and C. difficile. The Clostridium family of bacteria have been associated with a number of human maladies; aside from C. perfringens, as described above, another well-known and notable pathogen is C. difficile, the major causative agent for pseudomembraneous colitis and toxic megacolon as well as other antibiotic associated diarrheas (AAD).

C. difficile was first isolated in 1935 from intestinal flora of newborn infants (Hall et al., Am. J. Dis. Child. (1935) 49:390-402). In 1978, C. difficile was identified as the primary causative agent of pseudomembraneous colitis (now referred to as C. difficile associated diarrhea (CDAD) or C. difficile infection (CDI) (Bartlett et al., Gastroenterology (1978) 75:778-782), an inflammatory condition of the large intestine characterized by diarrhea that ranges in severity from mild to fulminant and is associated with the appearance of distinct raised plaques and neutrophil accumulation in the lumen of the intestinal lining. In general, these C. difficile related diarrheas result in about 10% to 30% mortality, especially in the elderly and in particular the elderly in hospital settings.

C. difficile has proven quite difficult to eradicate, especially in the hospital or healthcare setting (Loo et al., N. Engl. J Med. (2005) 353:2442-2449; Thomas et al., J Antimicrob Chemother (2003) 51:1339-1350). In fact, whereas only 1-3% of healthy adults are carriers of C. difficile, hospitalization increases the risk of colonization to as high as 50% in a manner directly proportional to the length of hospitalization (Bartlett and Perl, N. Engl. J Med. (2005) 353:2503-2505; Clabots et al., J Infect. Dis., 166, 561-567, 1992; McFarland et al., N. Engl. J Med., 320, 204-210, 1989). C. difficile infection is therefore a prevalent and growing problem within the healthcare industry.

There are few drugs that have shown promise in the treatment of CDI. Presently, only vancomycin (125 mg four times a day for a period of seven to fourteen days) is approved by the FDA for treatment of CDI. Metronidazole (250 mg three times a day for a period of seven to fourteen days) is also used extensively in clinical practice following early reports of its efficacy in CDI (Teasley et al., Lancet (1983) 2:1043-1046; Wilcox and Spencer, J. Hosp. Infect. (1992) 22:85-92). However, recent studies have noted relatively high and growing incidence of treatment failure and relapse following metronidazole therapy (Pepin et al., Clin. Infect. Dis. (2005) 40:1591-1597). Widespread vancomycin use in the treatment of CDI (as well as other more common infections) has raised concerns about selection for vancomycin resistant strains of C. difficile and other bacteria. These concerns have led to proposals for first-line metronidazole use, with vancomycin being reserved for patients who are severely ill or have failed prior therapy (Bartlett et al., supra) Overall, options for the treatment of CDI are limited.

Amino acyl tRNA synthetases represent a promising platform for the development of new antibacterial agents with little cross-resistance to currently marketed antibiotics (Hurdle et al., Antimicrob, Agents Chemother. (2005) 49:4821-33). These synthetases play an essential role in protein synthesis by charging tRNA molecules with their corresponding amino acid so that the amino acid can be delivered to the ribosome for protein synthesis. In most bacteria, including Clostridia such as C. perfringens and C. difficile, a decrease in the ratio of charged to uncharged tRNA triggers a physiological reaction called the “stringent response.” The stringent response induces a down-regulation of the synthesis of rRNA and tRNA, thereby inhibiting protein synthesis and ultimately the attenuation of bacterial growth. As such, amino acyl tRNA synthetases represent a potentially new molecular target for antibacterial agents. The inhibitor mupirocin (an inhibitor of isoleucyl tRNA synthetase) was released as a topical antibiotic in the treatment of S. aureus and S. pyogenes infections. Mupirocin is produced by the organism Pseudomonas fluorescens, and is an antibacterial agent used as the active ingredient in the product Bactroban®, marketed by GlaxoSmithKline.

This application describes compounds disclosed in U.S. Provisional Patent Application Ser. No. 60/826,957 entitled “Methods and Compositions For Treatment of Clostidium Based Infection,” filed Sep. 26, 2006, and incorporated by reference herein in its entirety; U.S. patent applications: ENANTIOMERIC COMPOUNDS WITH ANTIBACTERIAL ACTIVITY, Ser. No. 60/826,940, filed Sep. 26, 2006 and to corresponding US non-provisional and PCT applications filed on Sep. 11, 2007; SUBSTITUTED THIENOPYRIDONE COMPOUNDS WITH ANTIBACTERIAL ACTIVITY, Ser. No. 60/826,945 filed Sep. 26, 2006 and corresponding US non-provisional and PCT applications filed on Sep. 11, 2007; and SUBSTITUTED PHENYLETHER-THIENOPYRIDONE COMPOUNDS WITH ANTIBACTERIAL ACTIVITY, Ser. No. 60/826,954 filed Sep. 26, 2006 and corresponding US non-provisional and PCT applications filed on Sep. 11, 2007; U.S. Pat. No. 6,943,175, filed Dec. 5, 2003, U.S. Pat. No. 7,030,137, filed Feb. 27, 2004, and to U.S. patent application Ser. No. 10/729,416, filed Dec. 5, 2003 and Ser. No. 11/223,327, filed Sep. 9, 2005. Each of the above referenced applications and patents are incorporated by reference herein for all purposes.

Against this backdrop the present invention has been developed.

### DETAILED DESCRIPTION OF THE INVENTION

### Clostridium:

Clostridium is a spore-forming, anaerobic, Gram-positive bacillus. Clostridium genus members include common free-living bacteria as well as several important pathogens: Clostridium(C) perfringens, C. tetani, C. botulinum and C. difficile. C. perfringens is a common bacterium found in soil, often having a role in food poisoning and gas gangrene; C. tetani is the causative agent in tetanus or lockjaw (a disease largely eradicated in the industrialized world due to the tetanus vaccine); C. botulinum is the causative agent in botulism, found typically in soil or fish; and C. difficile is a bacterium associated with severe infections of the colon, showing an ability to flourish in the gut while other bacterium are eliminated during antibiotic treatment.

Methods and compounds of the invention are useful in the treatment of each of these Clostridium bacterium infections. However, because of its relative increasing prevalence in causation of disease, this case and its methods are directed toward autism spectrum disorder (ASD), and in particular, ASD cases caused by intestinal dysbiosis, and especially those cases caused by Clostridia, and especially those caused by Clostridia that belong to Clostridium clusters I, II and XI, including C. perfringens, C. botulinum, C. tetani, and C. difficile. Note, however, that inhibitors of the present invention are useful in the eradication and treatment of any of the Clostridium based infections.

C. difficile infection is a well-known intestinal disease caused by Clostridia (cluster XI). C. difficile infection results in extreme inflammation of the infected hosts' intestinal lining as caused by a group of secreted toxins. Clostridial toxins A and B (TcdA and TcdB) have been shown as the likely causative agents in this manner (Lyerly et al., Clin. Microbiol. Rev. (1988) 1:1-18; Voth et al., Clin. Microbiol. Rev. (2005) 18:247-363). TcdA and B are structurally and functionally related to glycosyltransferases which enter the intestinal epithelial cells by receptor-mediated endocytosis and catalyze UDP glucose-mediated glucosylation of small GTPases in the Ras superfamily (like Rho, Rac and Cdc42). Glucosylation of these Ras superfamily GTPases results in their irreversible inactivation and consequent actin condensation, cell rounding, membrane blebbing, disruptions of tight junctions between cells and ultimately cell death by apoptosis.

In the C. difficile genome, TcdA and B are encoded on a 19.6 kb pathogenicity locus (PaLoc). Also encoded on PaLoc are TcdC and D, the putative negative and positive regulators of TcdA and B expression. In addition, TcdE, a cell permeabilizing factor is encoded on PaLoc, a factor involved in the release of the two toxins.

In addition to TcdA and B, several strains of C. difficile encode a binary toxin encoded by cdtA and cdtB genes. These two genes are not encoded on the PaLoc. The proteins encoded by these genes, CDTa and CDTb, form a two-component toxin in which CDTb mediates receptor-mediated endocytosis and CDTa modifies actin filaments through its ADP-ribosyltransferase activity. CDTa and CDTb proteins are more than 80% identical in sequence with the corresponding components of the iota toxin from C. perfringens.

Vancomycin is the only antibiotic currently approved by the FDA for the treatment of C. difficile based infections. Metronidazole is also extensively used in clinical practice following early reports of its efficacy in CDI. However, recent studies have noted a relatively high and growing incidence of treatment failure and relapse following metronidazole therapy (Pepin et al., Clin Infect Dis (2005) 40:1591-1597). Widespread vancomycin use, however, raises concerns about selection for vancomycin being reserved for patients who are severely ill or have failed prior therapy.

Therefore, one aspect of the present invention is directed at developing new therapies for the treatment of Clostridium based infection. Compounds and methods of the present invention accomplish these interrelated goals.

Methionyl tRNA Synthetase:

Amino acyl tRNA synthetases represent a promising platform for the development of new antibacterial agents. These enzymes play an essential role in protein synthesis by charging tRNA molecules with their corresponding amino acid so that the ribosome can perform protein synthesis. In most bacteria, including pathogens, a decrease in the ratio of charged to uncharged tRNA triggers a physiological reaction called the “stringent response.” The stringent response induces a down-regulation of rRNA and tRNA, which results in the attenuation of bacterial growth.

Methionyl tRNA synthetase (MetRS) enzyme is a novel and unexploited target for treatments directed at Clostridium based infections, especially infections caused by C. difficile. Phylogenetic analysis of MetRS enzymes reveals that it falls into either a type I or type II class, where Gram-positive bacteria generally show a type I characteristic.

Therefore, the present invention targets MetRS enzymes. In preferred embodiments, the inhibitor compounds of the invention target MetRS enzymes and are particularly useful in the treatment of Clostridium, and more particularly, ASD caused by bacterial that belong to Clostridium clusters I, II and XI, including ASD caused by C. perfringens, C. botulinum, C. tetani, and C. difficile.

### MetRS Inhibitors:

The present invention provides inhibitors of Clostridium derived MetRS. Any inhibitor targeted at the MetRS enzyme is within the scope of the present invention, although a series of illustrative compounds are provided herein. The present invention teaches that inhibitors directed at the MetRS enzyme are extremely potent antibacterial agents in the treatment of Clostridium bacterium and in particular C. perfringens, C. botulinum, C. tetani, and C. difficile.

A number of potent inhibitors of Clostridium derived MetRS are provided. These compounds have been identified as potent antibacterial agents useful in the treatment of Clostridium infection, and in particular C. perfringens, C. botulinum, C. tetani, and C. difficile infection.

In brief, illustrative compounds of the invention show excellent anti-Clostridium activity as determined by IC₅₀, MIC₉₀ and animal study data (see Examples 2-5). In addition, compounds of the invention inhibit growth and production of C. difficile toxin (Example 6) and reduced C. difficile spore formation (Example 7). In particular, illustrative compounds of the present invention are surprisingly potent inhibitors of MetRS and of C. difficile growth and show the capability to treat animals having a C. difficile infection. MetRS inhibitors have good activity against C. difficile and C. perfringens but limited activity against other “friendly” intestinal strains (Citron et al., J. Antimicrob. Chemother. (2009) 63:972-976). The ability of MetRS inhibitors to inhibit spore formation and toxin production may be useful in reducing outbreaks, relapse and reinfection rates, including overgrowth of pathogenic Clostridium species following broad-spectrum antibiotic use in kids, such as antibiotics used for the treatment of chronic ear infections, that are known to be associated with the development of GI symptoms, and in some cases, ASD, including late-onset ASD. The combination of these benefits shows the utility of methods and compounds of the present invention.

In a number of the embodiments, inhibitors of the invention have a general structure as shown in formula (I):

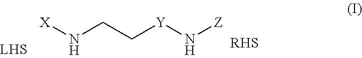

in which:
X is the left hand side (LHS) substituent and is a substituted or unsubstituted aryl or heteroaryl group;
Z is the right hand side (RHS) substituent and has a substituted or unsubstituted aryl or heteroaryl group; and
Y is a linker group having from one to six methylene groups in a straight chain and in which one or more methylene groups may have one or more (C₁₋₆) alkyl, (C₁₋₆)alkoxy or (C₁₋₆)alkylidenyl substituents.

Note that the linker:

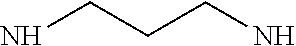

is preferred, providing an optimal spacing between the two aryl groups (noting that other similarly spaced linkers may be substituted).

In one embodiment, compounds of the present invention are shown in formula (II):

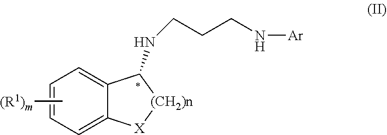

in which:
Ar is the right hand side (RHS) substituent and has a substituted or unsubstituted aryl or heteroaryl group;
X is selected from the group consisting of NH, O, S, SO, SO₂, or CH₂;
n is 1, 2 or 3;
* indicates an asymmetric carbon atom, wherein when n is 2 or 3, then * is R configuration; wherein when n is 1 and X is CH₂, then * is R configuration; and wherein when n is 1 and X is selected from the group consisting of NH, O, S, SO, or SO₂, then * is S configuration;
m is 0, 1, 2, 3, or 4; and
R¹ is independently selected from halo, cyano, hydroxyl, (C₁₋₆)alkyl (optionally substituted by halo, hydroxyl, amino, carboxy, or (C₁₋₆)alkoxycarbonyl), (C₃₋₇)cycloalkyl, C(₁₋₆)alkoxy, amino, mono- or di-(C₁₋₆)alkylamino, acylamino, carboxy, (C₁₋₆)alkoxycarbonyl, carboxy(C₁₋₆)alkyloxy, (C₁₋₆)alkylthio, (C₁₋₆)alkylsulphinyl, (C₁₋₆)alkylsulphonyl, sulphamoyl, mono- and di-(C₁₋₆)alkylsulphamoyl, carbamoyl, mono- and di-(C₁₋₆)alkylcarbamoyl and heterocyclic.

Preferred embodiments of the invention are those compounds of the formula (IIa) and (IIb):

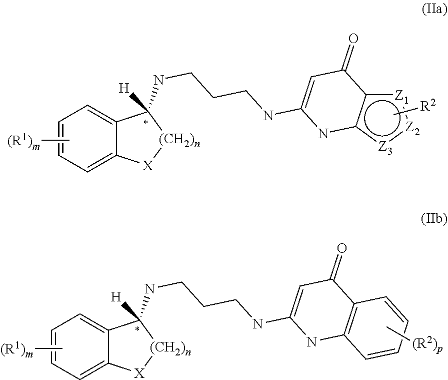

in which:
X is selected from the group consisting of NH, O, S, SO, SO₂, or CH₂;
n is 1, 2 or 3;
* indicates an asymmetric carbon atom, wherein when n is 2 or 3, then * is R configuration; wherein when n is 1 and X is CH₂, then * is R configuration; and wherein when n is 1 and X is selected from the group consisting of NH, O, S, SO, or SO₂, then * is S configuration;
R¹ is independently selected from halo, cyano, hydroxyl, (C₁₋₆)alkyl (optionally substituted by halo, hydroxyl, amino, carboxy, or (C₁₋₆)alkoxycarbonyl), (C₃₋₇)cycloalkyl, C(₁₋₆)alkoxy, amino, mono- or di-(C₁₋₆)alkylamino, acylamino, carboxy, (C₁₋₆)alkoxycarbonyl, carboxy(C₁₋₆)alkyloxy, (C₁₋₆)alkylthio, (C₁₋₆)alkylsulphinyl, (C₁₋₆)alkylsulphonyl, sulphamoyl, mono- and di-(C₁₋₆)alkylsulphamoyl, carbamoyl, mono- and di-(C₁₋₆)alkylcarbamoyl and heterocyclic;
m is 0, 1, 2, 3 or 4;
p is 0, 1, 2 or 3;
R² is independently selected from halo, cyano, hydroxyl, (C₁₋₆)alkyl (optionally substituted by halo, hydroxyl, amino, carboxy, or (C₁₋₆)alkoxycarbonyl), (C₃₋₇)cycloalkyl, C(₁₋₆)alkoxy, amino, mono- or di-(C₁₋₆)alkylamino, acylamino, carboxy, (C₁₋₆)alkoxycarbonyl, carboxy(C₁₋₆)alkyloxy, (C₁₋₆)alkylthio, (C₁₋₆)alkylsulphinyl, (C₁₋₆)alkylsulphonyl, sulphamoyl, mono- and di-(C₁₋₆)alkylsulphamoyl, carbamoyl, mono- and di-(C₁₋₆)alkylcarbamoyl and heterocyclic; and
when Z₁ is S, Z₂ and Z₃ are CH; when Z₂ is S, Z₁ and Z₃ are CH; and when Z₃ is S, Z₁ and Z₂ are CH.

Particularly preferred compounds of formula (IIa) and (IIb) include:
5-[3-((R)(−)-5,7-Dibromo-1,2,3,4-tetrahydro-naphthalen-1-ylamino)-propylamino]-4H-thieno[3,2-b]pyridine-7-one;
5-[3-((R)(+)-8-Bromo-6-chloro-chroman-4-ylamino)-propylamino]-4H-thieno[3,2-b]pyridine-7-one;
5-[3-((R)(+)-6,8-Dibromo-1,2,3,4-tetrahydro-quinolin-4-ylamino)-propylamino]-4H-thieno[3,2-b]pyridine-7-one;
5-[3-((R)(+)-6,8-Dibromo-chroman-4-ylamino)-propylamino]-4H-thieno[3,2-b]pyridine-7-one;
2-[3-((R)(+)-6,8-Dibromo-chroman-4-ylamino)-propylamino]-1H-quinolin-4-one; and
5-[3-((S)-5,7-Dibromo-benzofuran-3ylamino)-propylamino]-4H-thieno[3,2-b]pyridine-7-one.

It will be appreciated that certain compounds of the present invention may comprise one or more chiral centers so that compounds may exist as stereoisomers, including diastereoisomers and enantiomers. Embodiments of the invention cover all such stereoisomers, and mixtures thereof, including racemates and mixtures having an enantiomeric excess of one of the enantiomers.

Another embodiment of the present invention provides compounds of formula (III):

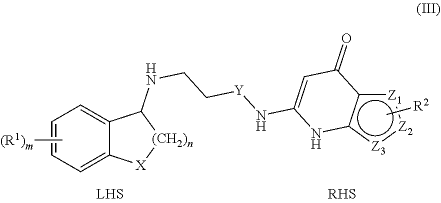

in which:
R¹ is selected from halo, cyano, hydroxyl, (C₁₋₆)alkyl (optionally substituted by halo, hydroxyl, amino, mono to perfluoro(C₁₋₃)alkyl, carboxy, or (C₁₋₆)alkoxycarbonyl), (C₃₋₇)cycloalkyl, (C₁₋₆)alkoxy, amino, mono- or di-(C₁₋₆)alkylamino, acylamino, carboxy, (C₁₋₆)alkoxycarbonyl, carboxy(C₁₋₆)alkyloxyl, (C₁₋₆)alkylthio, (C₁₋₆)alkylsulphanyl, (C₁₋₆)alkylsulphonyl, sulphamoyl, mono- and di(C₁₋₆)alkylsulphamoyl, carbamoyl, mono- and di-(C₁₋₆)alkylcarbamoyl, and heterocyclyl;
Y is a linker group having from one to six methylene groups in a straight chain and in which one or more methylene groups may have one or more (C₁₋₆) alkyl, (C₁₋₆)alkoxy or (C₁₋₆)alkylidenyl substituents;
R² is selected from halo, cyano, hydroxyl, (C₁₋₆)alkyl (optionally substituted by halo, hydroxyl, amino, mono to perfluoro(C₁₋₃)alkyl, carboxy, or (C₁₋₆)alkoxycarbonyl), (C₃₋₇)cycloalkyl, (C(₁₋₆)alkoxy, amino, mono- or di-(C₁₋₆)alkylamino, acylamino, carboxy, (C₁₋₆)alkoxycarbonyl, carboxy(C₁₋₆)alkyloxyl, (C₁₋₆)alkylthio, (C₁₋₆)alkylsulphanyl, (C₁₋₆)alkylsulphonyl, sulphamoyl, mono- and di(C₁₋₆)alkylsulphamoyl, carbamoyl, mono- and di-(C₁₋₆)alkylcarbamoyl, and heterocyclyl;
when Z₁ is S, Z₂ and Z₃ are CH; when Z₂ is S, Z₁ and Z₃ are CH; when Z₃ is S, Z₁ and Z₂ are CH;
X is NH, S, SO, SO₂, O or CH₂;
m is 0 or an integer from 1 to 4; and
n is one, two or three.

Preferred compounds of formula (III) include:
5-[3-(6,8-Dibromo-1,2,3,4-tetrahydro-quinolin-4-ylamino)-propylamino]-4H-thieno[3,2-b]pyridin-7-one;
5-[3-(6,8-Dibromo-chroman-4-ylamino)-propylamino]-4H-thieno[3,2-b]pyridin-7-one;
5-[3-(8-Bromo-6-chloro-chroman-4-ylamino)-propylamino]-4H-thieno[3,2-b]pyridin-7-one;
5-[3-(6-Chloro-8-iodo-chroman-4-ylamino)-propylamino]-4H-thieno[3,2-b]pyridin-7-one;
5-[3-(6-Bromo-8-chloro-chroman-4-ylamino)-propylamino]-4H-thieno[3,2-b]pyridin-7-one;
5-[3-(6-Bromo-8-chloro-1,2,3,4-tetrahydro-quinolin-4-ylamino)-propylamino]-4H-thieno[3,2-b]pyridin-7-one;
5-[3-(8-Bromo-6-chloro-1,2,3,4-tetrahydro-quinolin-4-ylamino)-propylamino]-4H-thieno[3,2-b]pyridin-7-one;
5-[3-(8-Bromo-6-methylsulfanyl-chroman-4-ylamino)-propylamino]-4H-thieno[3,2-b]pyridin-7-one; and
5-[3-(6-Bromo-8-fluoro-1,2,3,4-tetrahydro-quinolin-4-ylamino)-propylamino]-4H-thieno[3,2-b]pyridin-7-one.

Another embodiment of the invention provides compounds of formula (IV):

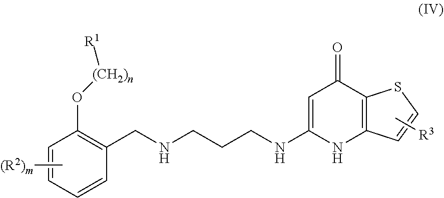

in which:
R¹ is selected from the group consisting of aryl and heteroaryl groups, including but not limited to substituted and unsubstituted benzene, toluene, phenol, anisole, thiazole, thiazolidine and pyridine, alkenes, imines, and other like substituents;
R² is independently selected from halo, cyano, hydroxyl, (C₁₋₆)alkyl (optionally substituted by halo, hydroxyl, amino, carboxy, or (C₁₋₆) alkoxycarbonyl), (C₁₋₆) cycloalkyl, (C₁₋₆) alkoxy, amino, mono- or di-(C₁₋₆)alkylamino, acylamino, carboxy, (C₁₋₆)alkoxycarbonyl, carboxy(C₁₋₆)alkyloxy, (C₁₋₆)alkylthio, (C₁₋₆)alkylsulphinyl, (C₁₋₆)alkylsulphonyl, sulphamoyl, mono- and di-(C₁₋₆)alkylsulphamoyl, carbamoyl, mono- and di-(C₁₋₆)alkylcarbamoyl, and heteroxy;
R³ is selected from a halo, (C₁₋₃)alkyl, (C₂₋₃)alkenyl, (C₂₋₃)alkynyl or other like substituents;
n is one, two or three; and
m is 0, 1, 2 or 3.

Preferred compounds of formula (IV) include:
5-{3-[3-Bromo-5-methylsulfanyl-2-(2-pyridin-3-yl-ethoxy)-benzylamino]-propylamino}-4H-thieno[3,2-b]pyridine-7-one;
5-(3-{3-bromo-5-methylsulfanyl-2-[2-(4-methyl-thiazol-5-yl)-ethoxyl]-benzylamino}-propylamino)-4H-thieno[3,2-b]pyridine-7-one;
5-[3-(3-bromo-5-methylsulfanyl-2-phenethyloxy-benzylamino)-propylamino]-4H-thieno[3,2-b]pyridine-7-one;
5-(3-{3,5-Dibromo-2-[2-(4-methyl-thiazol-5-yl)-ethoxyl]-benzylamino}-propylamino)-4H-thieno[3,2-b]pyridine-7-one;
5-{3-[3,5-Dibromo-2-(2-pyridin-3-yl-ethoxy)-benzylamino]-propylamino}-4H-thieno[3,2-b]pyridine-7-one;
5-(3-{3,5-Dibromo-2-[2-(4,5-dimethyl-thiazol-2-yl)-ethoxy]-benzylamino}-propylamino)-4H-thieno[3,2-b]pyridine-7-one;
5-[3-(3,5-Dibromo-2-phenethyloxy-benzylamino)-propylamino]-4H-thieno[3,2-b]pyridine-7-one;
5-{3-[3,5-Dibromo-2-(3-pyridin-3-yl-propoxy)-benzylamino]-propylamino}-4H-thieno[3,2-b]pyridine-7-one;
5-(3-{3,5-Dibromo-2-[2-(3,4-dichloro-phenyl)-ethoxy]-benzylamino}-propylamino)-4H-thieno[3,2-b]pyridine-7-one;
5-(3-{3,5-Dibromo-2-[2-(4-methoxy-phenyl)-ethoxy]-benzylamino}-propylamino)-4H-thieno[3,2-b]pyridine-7-one;
5-{3-[3,5-Dibromo-2-(2-p-tolyl-ethoxy)-benzylamino]-propylamino}-4H-thieno[3,2-b]pyridine-7-one;
5-3-{3,5-Dibromo-2-[2-(fluoro-phenyl)-ethoxy]-benzylamino}-propylamino)-4H-thieno[3,2-b]pyridine-7-one;
5-(3-{3,5-Dibromo-2-[2-(4-chloro-phenyl)-ethoxy]-benzylamino}-propylamino)-4Hthieno[3,2-b]pyridine-7-one; and
5-{3-[3-Bromo-5-methylsulfanyl-2-(3-pyridin-3-yl-propoxy)-benzylamino]-propylamino}-4H-thieno[3,2-b]pyridine-7-one.

Another embodiment of the invention provides compounds of formula (V):

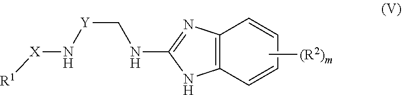

in which:
R¹ is an optionally substituted aryl or an optionally substituted heteroaryl ring;
R² is independently selected from halo, cyano, hydroxyl, (C₁₋₆)alkyl, (optionally substituted by halo, hydroxy, amino, mono to perfluoro(C₁₋₃)alkyl, carboxy, or (C₁₋₆)alkoxycarbonyl), (C₃₋₇)cycloalkyl, (C₁₋₆)alkoxy, amino, mono- or di-(C₁₋₆)alkylamino, acylamino, carboxy, (C₁₋₆)alkoxycarbonyl, carboxy(C₁₋₆)alkyloxy, (C₁₋₆)alkylthio, (C₁₋₆)alkylsulphinyl, (C₁₋₆)alkylsulphonyl, sulphamoyl, mono- and di(C₁₋₆)alkylsulphamoyl, carbamoyl, mono- and di-(C₁₋₆)alkylcarbamoyl, and heterocyclyl;
m is 0, 1, 2, or 3;
X is CH₂ or CHR³ in which R³ is C(₁₋₆)alkyl or is linked to the ortho position of an aryl or heteroaryl ring of R¹ to form a 5 to 7 membered ring optionally including oxygen or nitrogen as a ring atom; and
Y is (C₁₋₃)alkylene or (C₄₋₆)cycloalkylene.

Another embodiment of the invention provides compounds of formula (VI):

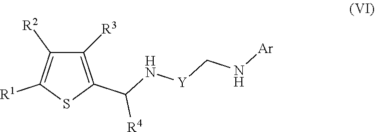

in which:
R¹ is selected from the group consisting of Br, optionally fluoro-substituted C(₁₋₃)alkyl, optionally fluoro-substituted (C₂₋₃)alkenyl, and (C₂₋₃)alkyl;
R² is a halogen, preferably Br;
R³ is selected from the group consisting of (C₁₋₃)alkyl, (C₂₋₅)alkenyl, (C₂₋₃)alkynyl;
R⁴ is selected from the group consisting of H, and (C₁₋₃)alkyl;
Y is C(₁₋₃)alkyl; and
Ar is selected from the group consisting of substituted or unsubstituted heteroaryl imidiazole, substituted or unsubstituted quinolone, substituted or unsubstituted benzimidazole, substituted or unsubstituted fused heteroaryl pyridone, substituted or unsubstituted fused aryl pyrimidone, or substituted or unsubstituted fused heteroaryl pyrimidone.

Another embodiment of the invention provides compounds of formula (VII):

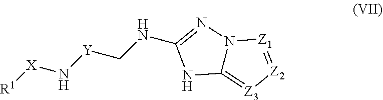

in which:
R¹ is an optionally substituted aryl or an optionally substituted heteroaryl ring;
X is CH₂ or CHR³ in which R³ is C(₁₋₆)alkyl or is linked to the ortho position of an aryl or heteroaryl ring of R¹ to form a 5 to 7 membered ring optionally including oxygen or nitrogen as a ring atom;
Y is C(₁₋₃)alkylene or C(₄₋₆)cycloalkylene; and
Z₁, Z₂ and Z₃ is each independently selected from N or CR₄ in which R₄ is hydrogen or a substituent selected from halogen, cyano, (C₁₋₆)alkyl, mono- to per-fluoro(C₁₋₃)alkyl, (C₃₋₇)cycloalkyl, (C₂₋₆)alkenyl, (C₁₋₆)alkoxy, (C₂₋₆)alkenoxy, arylC(₁₋₆)alkoxy, halo(C₁₋₆)alkyl, hydroxyl, amino, mono- or di-(C₁₋₆)alkylamino, acylamino, nitro, carboxy, (C₁₋₆)alkoxycarbonyl, (C₁₋₆)alkenyloxycarbonyl, (C₁₋₆)alkoxycarbonyl(C₁₋₆)alkyl, carboxy(C₁₋₆)alkyl, (C₁₋₆)alkylcarbonyloxy, carboxy(C₁₋₆)alkyloxy, (C₁₋₆)alkoxycarbonyl(C₁₋₆)alkoxy, C(₁₋₆)alkylthio, (C₁₋₆)alkylsulphinyl, (C₁₋₆)alkylsulphonyl, sulphamoyl, mono- and di-(C₁₋₆)-alkylsulphamoyl, carbamoyl, mono- and di-(C₁₋₆)alkylcarbamoyl, and heterocyclyl.

Another embodiment of the invention provides compounds of formula (VIII):

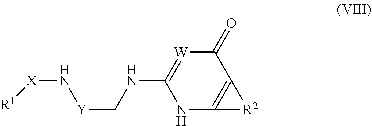

in which:
W is CH and R² is the residue of a 5 or 6-membered heteroaryl ring, or W is N and R² is the residue of an 5 or 6-membered heteroaryl ring or an aryl ring, which heteroaryl or aryl ring is optionally substituted with from one to three substituents selected from halo, cyano, hydroxyl, (C₁₋₆)alkyl (optionally substituted by halo, hydroxyl, amino, mono, to perfluoro(C₁₋₃)alkyl, carboxy, or (C₁₋₆)alkoxycarbonyl, (C₃₋₇)cycloalkyl, C(₁₋₆)alkoxy, amino, mono- or di-(C₁₋₆)alkoxycarbonyl, acylamino, carboxy, (C₁₋₆)alkoxycarbonyl, carboxy(C₁₋₆)alkyloxy, (C₁₋₆)alkythio, (C₁₋₆)alkylsulphamoyl, carbamoyl, mono- and di-(C₁₋₆)alkycarbamoyl, and heterocyclyl;
R¹ is an optionally substituted aryl or an optionally substituted heteroaryl ring;
X is CH₂ or CHR³ in which R³ is C(₁₋₆)alkyl or R³ may be linked to the ortho position or an aryl or heteroaryl ring of R¹ to form a 5 to 7 membered ring optionally including oxygen or nitrogen as a ring atom; and
Y is C(₁₋₃)alkylene or C(₄₋₆)cycloalkylene.

Another embodiment of the invention provides compounds of formula (IX):

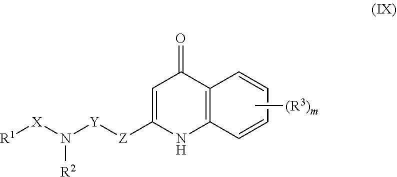

in which:
R¹ is optionally substituted aryl or optionally substituted heteroaryl group;
R² is hydrogen, C(₁₋₆)alkyl, aryl C(₁₋₄)alkyl, aryl C(₂₋₄)alkenyl or C(₁₋₆)alkylcarbonyl;
R³ is selected from halo, cyano, hydroxyl, (C₁₋₆)alkyl (optionally substituted by halo, hydroxyl, amino, mono to perfluoro(C₁₋₃)alkyl, carboxy, or (C₁₋₆)alkoxycarbonyl), (C₃₋₇)cycloalky, C(₁₋₆)alkoxy, amino, mono- or di-(C₁₋₆)alkoxycarbonyl, acylamino, carboxy, (C₁₋₆)alkoxycarbonyl, carboxy(C₁₋₆)alkyloxy, (C₁₋₆)alkythio, (C₁₋₆)alkylsulphamoyl, carbamoyl, mono- and di-(C₁₋₆)alkycarbamoyl, and heterocyclyl;
m is 0 or an integer from one to three;
X is CHR⁴ wherein R⁴ is hydrogen, C(₁₋₆)alkyl or aryl, C(₂₋₄)alkylene, C(₃₋₄)alkenylene or CO;
Y is a linker group having from two to six methylene groups in a straight chain and in which one or more methylene groups may have one or more C(₁₋₆)alkyl, C(₁₋₆)alkoxy, or C(₁₋₆)alkylidenyl substituents and in which chain 1,2- or 1,3-carbon atoms may be linked by a C(₂₋₃)alkyene or C₃ alkenylene bridge;
R¹ and X or R¹ and R² may be linked by a polymethylene chain to form a 5 to 7 membered ring, optionally substituted by C(₁₋₆)alkyl;
X and R², X and Y or Y and R² may be linked by a polymethylene chain to form a 4 to 7 membered ring, optionally substituted by C(₁₋₆)alkyl; and
Z is NH or O.

Finally, another embodiment of the invention provides compounds of formula (X):

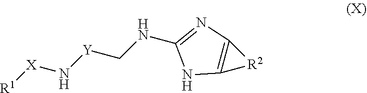

in which:
R¹ is an optionally substituted aryl or an optionally substituted heteroaryl ring;
R² is the residue of a 5 or 6-membered heteroaryl ring which is optionally substituted with from 1 to 3 substituents selected from halo, cyano, hydroxy, (C₁₋₆)alkyl (optionally substituted by halo, hydroxy, amino, mono to perfluoro(C₁₋₃)alkyl, carboxy or (C₁₋₆)alkoxycarbonyl), (C₃₋₇)cycloalkyl, (C₁₋₆)alkoxy, amino, mono- or di-(C₁₋₆)alkylamino, acylamino, carboxy, (C₁₋₆)alkoxycarbonyl, carboxy(C₁₋₆)alkyloxy, (C₁₋₆)alkylthio, (C₁₋₆)alkylsulphinyl, (C₁₋₆)alkylsulphonyl, sulphamoyl, mono- and di-(C₁₋₆)alkylsulphamoyl, carbamoyl, mono- and di-(C₁₋₆)alkylcarbamoyl, and heterocyclyl;
X is CH₂ or CHR³ in which R³ is C(₁₋₆)alkyl or is linked to the ortho position of an aryl or heteroaryl ring of R¹ to form a 5 to 7 membered ring optionally including oxygen or nitrogen as a ring atom; and
Y is C(₁₋₃)alkylene or C(₄₋₆)cycloalkylene.

Compounds of the invention can also be salts of the compounds shown in formulas (I)-(X). Salts may be formed from inorganic and organic acids. Representative examples of suitable inorganic and organic acids from which pharmaceutically acceptable salts of compounds of formulas (I)-(X) may be formed include: maleic, fumaric, benzoic, ascorbic, pamoic, succinic, bismethylene-salicylic, methanesulfonic, ethanedislufonic, acetic, propionic, tartaric, salicylic, citric, gluconic, aspartic, stearic, palmitic, itaconic, glycolic, p-aminobenzoic, glutamic, benzenesulfonic, hydrochloric, hydrobromic, sulfuric, cyclohexylsulfamic, phosphoric and nitric acids.

When used herein, the term “alkyl” and similar terms such as “alkoxy” includes all straight chain and branched isomers. Representative examples thereof include methyl, ethyl, n-propyl, iso-propyl, n-butyl, sec-butyl, iso-butyl, t-butyl, n-pentyl and n-hexyl.

When used herein, the terms “alkenyl” and “alkynyl” include all straight chain and branched isomers. Representative examples thereof include vinyl, ethynyl and 1-propynyl.

Preferred substituents for alkyl and alkenyl groups include, for example, and unless otherwise defined, halogen, cyano, azido, nitro, carboxy, (C₁₋₆)alkoxycarbonyl, carbamoyl, mono- or di-(C₁₋₆)alkylcarbamoyl, sulpho, sulphamoyl, mono- or di-(C₁₋₆)alkylsulphamoyl, amino, mono- or di-(C₁₋₆)alkylamino, acylamino, ureido, (C₁₋₆)alkoxycarbonylamino, 2,2,2-trichloroethoxycarbonylamino, aryl, heterocyclyl, hydroxy, (C₁₋₆)alkoxy, acyloxy, oxo, acyl, 2-thienoyl, (C₁₋₆)alkylthio, (C₁₋₆)alkylsulphinyl, (C₁₋₆)alkylsulphonyl, hydroxyimino, (C₁₋₆)alkoxyimino, hydrazino, hydrazono, benzohydroximoyl, guanidino, amidino and iminoalkylamino.

When used herein, the term “aryl” includes, unless otherwise defined, phenyl or naphthyl optionally substituted with up to five, preferably up to three substituents.

When substituted, an aryl group may have up to three substituents. Preferred substituents for an aryl group include, for example, and unless otherwise defined, halogen, cyano, (C₁₋₆)alkyl, mono to perfluoro(C₁₋₃)alkyl, (C₃₋₇)cycloalkyl, (C₂₋₆)alkenyl, (C₁₋₆)alkoxy, (C₂₋₆)alkenoxy, arylC(₁₋₆)alkoxy, halo(C₁₋₆)alkyl, hydroxy, amino, mono- or di-(C₁₋₆)alkylamino, acylamino, nitro, carboxy, (C₁₋₆)alkoxycarbonyl, (C₁₋₆)alkenyloxycarbonyl, (C₁₋₆)alkoxycarbonyl(C₁₋₆)alkyl, carboxy(C₁₋₆)alkyl, (C₁₋₆)alkylcarbonyloxy, carboxy(C₁₋₆)alkyloxy, (C₁₋₆)alkoxycarbonyl(C₁₋₆)alkoxy, (C₁₋₆)alkylthio, (C₁₋₆)alkylsulphinyl, (C₁₋₆)alkylsulphonyl, sulphamoyl, mono- and di-(C₁₋₆)-alkylsulphamoyl, carbamoyl, mono- and di-(C₁₋₆)alkylcarbamoyl, and heterocyclyl.

When used herein, the term “heteroaryl” includes single or fused rings comprising up to four hetero-atoms in the ring selected from oxygen, nitrogen and sulphur. Preferably the heteroaryl ring comprises from 4 to 7, preferably 5 to 6, ring atoms. A fused heteroaryl ring system may include carbocyclic rings and need only include one heterocyclic ring.

When used herein, the term “heterocyclyl” includes aromatic and non-aromatic single or fused rings comprising up to four hetero-atoms in the ring selected from oxygen, nitrogen and sulphur. Suitably the heterocyclic ring comprises from 4 to 7, preferably 5 to 6, ring atoms. A fused heterocyclic ring system may include carbocyclic rings and need only include one heterocyclic ring.

When substituted, a heteroaryl or a heterocyclyl group may have up to three substituents. Preferred such substituents include those previously mentioned for an aryl group as well as oxo.

When used herein, the terms “halogen” and “halo” include fluorine, chlorine, bromine, and iodine and fluoro, chloro, bromo, and iodo, respectively.

The compounds of the present invention are suitably provided in substantially pure form, for example at least 50% pure, suitably at least 60% pure, advantageously at least 75% pure, preferably at least 85% pure, more preferably at least 95% pure, especially at least 98% pure. All percentages are calculated on a weight/weight basis. All impure or less pure forms of a compound according to the invention may, for example, be used in the preparation of more pure forms of the same compound or of a related compound (for example a corresponding derivative) suitable for pharmaceutical use.

Each of the compounds having formulas (I)-(X) are potent inhibitors of MetRS and have shown potent antibacterial activity against Clostridium based infections, and in particular against C. difficile based infections. Further, the compounds of the present invention are specific for bacterial MetRS and show little or no activity against mammalian MetRS, a good feature for use as an antibacterial agent.

A “compound of the invention” in context of the present invention means any compound having activity that possesses an IC₅₀<64 μM, an MIC₉₀<16 μg/mL, or an increased survival in an in vivo Clostridium difficile infection setting. In preferred embodiments the compounds of the invention have a structure as shown in formula (I) and in more preferred embodiments the compounds of the invention have a structure as shown in formulas (II)-(X) or of one of the compounds as described in one of the incorporated references herein.

The compounds of the present invention can be prepared by methods as described in U.S. Pat. Nos. 6,943,175, 7,973,050, 7,994,192, 8,697,720, or in one of the references that have been incorporated by reference.

### BRIEF DESCRIPTION OF THE DRAWINGS

FIG. 1 illustrates the impact of CRS3123 on major phyla of the intestinal flora in healthy volunteers in a multiple ascending dose Phase 1b clinical trial.

### METHODS FOR AUTISM SPECTRUM DISORDER TREATMENT

Compounds of this invention (enumerated or incorporated herein by reference) are active against Clostridium bacterium, such as C. perfringens, C. tetani, C. botulinum and C. difficile. In preferred aspects, the compounds of the invention are particularly active against C. perfringens and C. difficile, and can be used in relation to antibiotic-resistant strains of these bacteria.

Accordingly, the present invention provides a method of treating ASD in mammals and in some embodiments humans, which method comprises administering a therapeutically effective amount of a compound of the invention to a mammal in need of such therapy. A therapeutically effective amount of a compound is an amount that will elicit a biologic or medical response in the mammal that is being treated by a healthcare professional (including doctors, nurses, physician assistants, veterinarians, etc.). Note that the term mammal refers to any warm-blooded animal of the class Mammalia, including human, farm and domesticated animals, such as sheep, goat, cows, poultry, dog, cat, horse, etc.

In another aspect of the invention, methods are provided for treating ASD in a mammal which methods comprise administering a prophylactically effective amount of a compound of the invention to the mammal in need of such therapy. A prophylactically effective amount of a compound of the invention is an amount that will prevent or inhibit affliction or mitigate affliction of a mammal with ASD.

The present invention provides a pharmaceutical composition comprising a compound of the invention together with a pharmaceutically acceptable carrier or excipient. In preferred embodiments the pharmaceutical composition comprises a compound of formula (I) together with a pharmaceutically acceptable carrier or excipient and in more preferred embodiments the pharmaceutical composition comprises a compound of formula (II), (IIa), (IIb), (III), (IV), (V), (VI), (VII), (VIII), (IX), or (X) together with a pharmaceutically acceptable carrier or excipient.

The present invention further provides pharmaceutical compositions comprising combinations of two or more compounds of the invention together with a pharmaceutically acceptable carrier or excipient. For example, a pharmaceutical composition of the invention can include a compound of formula (I) and a compound of formula (II) in combination with the carrier or excipient. In addition, pharmaceutical compositions of the present invention can include other known antibiotic agents, for example, vancomycin or metronidazole. In addition, pharmaceutical compositions of the present invention can include other known agents, such as those that bind to toxins produced by Clostridium bacteria, including those produced by C. perfringens, C. botulinum, C. tetani, and C. difficile.

The present invention provides a method of treating ASD in mammals, especially in humans and in domesticated mammals, which comprises administering a compound of the invention, or a composition according to the invention, to a patient in need thereof.

The invention further provides the use of compounds of the invention in the preparation of a medicament composition for use in the treatment of ASD.

The compounds and compositions according to the invention may be formulated for administration in any convenient way for use in human or veterinary medicine, by analogy with other antibiotics.

The compounds and compositions according to the invention may be formulated for administration by any route, for example oral, topical, parenteral, or rectal. The compositions may, for example, be made up in the form of tablets, capsules, powders, granules, lozenges, creams, suppositories, syrups, or liquid preparations, for example solutions or suspensions, which may be formulated for oral use or in sterile form for parenteral administration by injection or infusion.

Tablets and capsules for oral administration may be in unit dosage form, and may contain conventional excipients including, for example, binding agents, for example, syrup, acacia, gelatin, sorbitol, tragacanth, or polyvinylpyrrollidone; fillers, for example lactose, sucrose, maize-starch, calcium phosphate, sorbitol or glycine; tabletting lubricants, for example magnesium stearate, talc, polyethylene glycol or silica; disintegrants, for example potato starch; and pharmaceutically acceptable wetting agents, for example sodium lauryl sulphate. The tablets may be coated according to methods well known in normal pharmaceutical practice.

Oral liquid preparations may be in the form of, for example, aqueous or oily suspensions, solutions, emulsions, syrups or elixirs, or may be presented as a dry product for reconstitution with water or another suitable vehicle before use. Such liquid preparations may contain conventional additives, including, for example, suspending agents, for example sorbitol, methyl cellulose, glucose syrup, gelatin, hydroxyethyl cellulose, carboxymethyl cellulose, aluminum stearate gel or hydrogenated edible fats; emulsifying agents, for example lecithin, sorbitan monooleate or acacia; non-aqueous vehicles (which may include edible oils), for example almond oil, oily esters (for example glycerine), propylene glycol, or ethyl alcohol; preservatives, for example methyl or propyl p-hydroxybenzoate or sorbic acid; and, if desired, conventional flavoring and color agents.

Compositions according to the invention intended for topical administration may, for example, be in the form of ointments, creams, lotions, eye ointments, eye drops, ear drops, impregnated dressings, and aerosols, and may contain appropriate conventional additives, including, for example, preservatives, solvents to assist drug penetration, and emollients in ointments, gels, and creams. Such topical formulations may also contain compatible conventional carriers, for example cream or ointment bases, and ethanol or oleyl alcohol for lotions. Such carriers may constitute from about 1% to about 98% by weight of the formulation; more usually they will constitute up to about 80% by weight of the formulation.

Compositions according to the invention may be formulated as suppositories, which may contain conventional suppository bases, for example cocoa-butter or other glycerides.

Compositions according to the invention intended for parenteral administration may conveniently be in fluid unit dosage forms, which may be prepared utilizing the compound and a sterile vehicle, water being preferred. The compound, depending on the vehicle and concentration used, may be either suspended or dissolved in the vehicle. In preparing solutions, the compound may be dissolved in water for injection and filter-sterilized before being filled into a suitable vial or ampoule, which is then sealed. Advantageously, conventional additives including, for example, local anesthetics, preservatives, and buffering agents can be dissolved in the vehicle. In order to enhance the stability of the solution, the composition may be frozen after being filled into the vial, and the water removed under vacuum; the resulting dry lyophilized powder may then be sealed in the vial and an accompanying vial of water for injection may be supplied to reconstitute the liquid prior to use. Parenteral suspensions may be prepared in substantially the same manner except that the compound is suspended in the vehicle instead of being dissolved and sterilization cannot be accomplished by filtration. The compound may instead be sterilized by exposure to ethylene oxide before being suspended in the sterile vehicle. Advantageously, a surfactant or wetting agent is included in such suspensions in order to facilitate uniform distribution of the compound.

A compound or composition according to the invention may suitably be administered to the patient in an effective amount to treat ASD or prophylactic amount.

A composition according to the invention may suitably contain from 0.1% by weight, preferably from 10 to 60% by weight, of a compound according to the invention (based on the total weight of the composition), depending on the method of administration.

The compounds according to the invention may suitably be administered to the patient at a daily dosage of from 1.0 to 100 mg/kg of body weight. For an adult human (of approximately 70 kg body weight), from 50 to 3000 mg, for example about 1500 mg, of a compound according to the invention may be administered daily. Suitably, the dosage for adult humans is from 2 to 40 mg/kg per day. Higher or lower dosages may, however, be used in accordance with normal clinical practice.

When the compositions according to the invention are presented in unit dosage form, each unit dose may suitably comprise from 25 to 1000 mg, preferable from 50 to 500 mg, of a compound according to the invention.

### Packs and Kits:

The invention also provides pharmaceutical packs or kits comprising one or more containers having one or more of the compounds of the invention. In addition, packs or kits can include instructions for use and any appropriate notices by a governmental agency regulating the manufacture, use or sale of the products. It is also envisioned that packs and kits of the invention can include other biologic agents useful in the treatment of Clostridium based infection.

### EXAMPLES

The following examples are provided for illustrative purposes only and are not intended to limit the scope of the invention.

### Example 1: Compounds of the Present Invention have Potent Antibacterial Activity Against C. difficile and C. perfringens

MetRS inhibitor compounds were also assayed for their capacity to inhibit C. difficile and C. perfringens growth. MIC₉₀ (minimum inhibition concentration required to inhibit the growth of 90% of C. difficile) was determined using standard agar based assays according to CLSI.

Organisms:

All compounds were tested for antibacterial activity against a collection of non-repeat clinical isolates of C. difficile and C. perfringens. The organisms were stored frozen in Brucella broth supplemented with 20% glycerol. The organisms were retrieved from the freezer and subcultured twice onto CDC agar to ensure purity and growth. The plates were incubated under anaerobic conditions for at least 24 hours. Bacterial colonies were examined for morphology; yellow color, ground glass texture and characteristic odor. The control organism tested was Bacteroides fragilis ATCC 25285.

Antimicrobial Susceptibility Testing:

Antimicrobial susceptibility testing was conducted by the agar dilution method on Brucella agar supplemented with vitamin K, hemin and 5% laked sheep blood in accordance with CLSI guidelines (CLSI, M11-A2). The test compounds were serially diluted and added to molten supplemented Brucella agar. Drug free plates were inoculated before and after inoculation of each antimicrobial plate series and were used as growth controls. Anaerobic/aerobic growth controls were conducted on drug free plates after two sets of drug plates. Bacterial colonies were suspended in Brucella broth to a turbidity equal to that of a 0.5 McFarland standard and applied to a plate with a Steers replicator that delivered 10⁵ CFU/spot. The plates were incubated under anaerobic conditions for 24 hours at 35° C. prior to the reading of the results. The minimum inhibitory concentration (MIC) was the concentration that completely inhibited growth or caused a marked reduction in the appearance of growth compared to that of the drug-free growth control.

Results:

MIC₉₀ for MetRS inhibitor compounds ranged from 0.25 to >32 μg/ml for all organisms tested in this study (Citron et al. (2009) supra). These results indicate the potent activity of the compounds of the present invention against C. difficile, typically around 0.5 g/ml, and an MIC₉₀ value of 1.0 μg/ml for a representative MetRS inhibitor CRS3123 (previously designated as REP3123, Citron et al., (2009) supra). Similarly, CR3123 exhibited potent activity against C. perfringens, with an MIC₉₀ value of 0.25 μg/ml (Citron et al., (2009) supra). In addition, IC₅₀ data indicates that the compounds of the present invention are specific for C. difficile, showing little or no activity against mammalian MetRS. MetRS inhibitor compounds show potent activity against C. difficile and Gram-positive bacteria while sparing normal gut flora. It is relevant to note in this context that members of Clostridium genus show strong MetRS sequence identity (U.S. Pat. No. 8,658,670).

### Example 2: Effects on Animal Models

We have shown previously that CRS3123 is active in the hamster model of C. difficile infection (Ochsner et al. J. Antimicrob. Chemother. (2009) 63:964-71). Animal models for ASD have also been developed. A number of ASD models utilize mice in which specific genes related to synaptic function have been disrupted (Shinoda et al., Exp. Anim. (2013) 62:71-78). Such models are useful for determining the role of specific genes in autism, however, because the genetic basis of autism is at best poorly understood, their general utility is questionable. For ASD that may be caused by environmental factors, a more appropriate model is the maternal immune activation (MIA) model, which was developed based on the epidemiological observation that infection during pregnancy is associated with an increase in the risk of autism in offspring. In this model, pregnant dams are treated by intraperitoneal injection with polyinosinic-polycytidylic acid, or poly(I:C), which is a double-stranded RNA mimetic known to activate the innate immune response through the toll-like receptor 3 (TLR3) pathway in a manner that simulates viral infection. The offspring exhibit behavioral, communication and anxiety-like abnormalities similar to ASD in humans. The MIA model has been used recently to demonstrate that the MIA mice exhibit an increase in gut permeability as well as dysbiosis typically observed in autistic kids. In proof-of-principle experiments, partial restoration of intestinal flora with specific treatments, such as with oral delivery of Lactobacillus reuteri (Buffington et al. Cell (2016) 165:1762-75) or Bacteroides fragilis (Hsiao et al. (2013), supra), has been shown to alleviate some of the ASD symptoms in MIA mice. In this context, we note that CRS3123 delivered orally to healthy volunteers in a repeat-dose phase 1b clinical trial (vide infra), shows minimal effects on the composition of normal flora at the 200 mg BID dose, which is consistent with its narrow-spectrum and limited activity against most constituents of the normal intestinal flora. At the 400 mg BID dose, and especially at the 600 mg BID dose, CRS3123 exhibits an increase in the proportion of Bacteroidetes, among which B. fragilis is one of the major members. Since B. fragilis has been shown to be able to reduce the severity of autism symptoms in the MIA model (Hsiao et al., (2013) supra), treatment of MIA mice with CRS3123, as well as with other MetRS inhibitors described herein, is expected to be similarly effective.

### Example 3: Safety and Tolerability in Phase 1 Clinical Testing in Healthy Human Volunteers

### Single Ascending Dose Phase 1a Study

CRS3123 has been evaluated in a first in human Phase 1 trial sponsored by the Division of Microbiology and Infectious Diseases (DMID) at the National Institute of Allergy and Infectious Diseases (DMID 10-0008) entitled “Randomized, Double-Blind, Placebo-Controlled, Single Ascending Dose Phase I Trial to Determine the Safety and Pharmacokinetics of CRS3123 Administered Orally to Healthy Adults”. The study was performed at the Johns Hopkins University. The primary objective of the study was to determine the safety and tolerability of escalating doses of CRS3123 following a single oral administration to healthy fasted subjects. The secondary objective was to assess the plasma PK characteristics of CRS3123 after a single oral dose. Forty healthy male and female subjects, ages 18 to 45, were randomized into five cohorts at the following dose levels: 100, 200, 400, 800 and 1200 mg. Per cohort, six subjects received CRS3123, and two received placebo.

A low but detectable fraction of the CRS3123 appeared to be absorbed at each of the doses investigated. CRS3123 appeared to be metabolized in the body via glucuronidation based upon mass spectral analysis of the eluting peaks. Due to the presence of two metabolites in the main eluting peak, elimination of the parent compound could not be measured. Some of the parent drug and its metabolites were excreted into the urine. Systemic exposure did not appear to be dose-proportional but this observation may have been affected by the multiple components comprising the main peak. In the absence of analytical standards for the metabolites, the data suggest the generation of three glucuronide species during metabolism of the parent compound.

Similar percentages of CRS3123-treated subjects (93.3%) and placebo-treated subjects (90.0%) reported treatment-emergent adverse events (TEAEs). Also, there were similar frequencies of TEAEs for CRS3123 treated subjects per cohort and across cohorts. There were no deaths, SAEs, or severe or life-threatening TEAEs noted in the CRS3123-treated subjects. Percentages of subjects with TEAEs in the CRS3123 cohorts did not correlate with dose of study drug.

For the CRS3123-treated subjects (combined), TEAEs with the highest frequency (≥5%) were decreased hemoglobin (23.3%), headache (20.0%), abnormal urine analysis (20.0%), and positive urine leukocyte esterase (16.7%). The majority of the TEAEs reported for both CRS3123-treated and placebo-treated subjects were classified as mild (overall, 92.6%). Moderate TEAEs were reported in 7.4% CRS3123-treated subjects versus 3.7% placebo-treated subjects. In addition, there was one severe TEAE in one placebo-treated subject, a Grade 3 proteinuria that resolved without intervention. The incidence of these TEAEs did not correlate with dose of study drug. There were no severe TEAEs among the CRS3123-treated subjects. Approximately half the TEAEs reported in both the CRS3123-treated and the placebo-treated subjects were related to study drug. For TEAEs related to CRS3123 treatment, the highest percentages of occurrence (combined) were headache (20.0%), decreased hemoglobin (16.7%), abnormal urine analysis (16.7%), and decreased blood calcium (13.3%). For TEAEs related to placebo treatment (combined), the highest percentage of distribution was abnormal urine analysis (20%).

The results of this Phase 1 study demonstrate the safety and tolerability of CRS3123 at the escalating single doses tested in a group of healthy adult subjects. While no analytical standards exist for measuring possible metabolites, the data suggest the generation of three glucuronide species during metabolism of CRS3123. Overall, CRS3123 was safe and well-tolerated in this single dose study.

### Multiple Ascending Dose Phase 1b Study

The multiple ascending dose Phase 1 study of CRS3123 entitled “Randomized, Double-Blind, Placebo-Controlled, Multiple Ascending Dose Phase 1 Trial to Determine the Safety and Pharmacokinetics of CRS3123 Administered Orally to Healthy Adults” was also sponsored by DMID (Protocol No. 10-0009). The study was performed at Quintiles Phase I Services in Overland Park, KS. The primary objective of the study was to determine the safety and tolerability of escalating doses of CRS3123 administered twice daily for ten days to healthy subjects. The secondary objective was to determine the plasma pharmacokinetic characteristics of CRS3123 after multiple oral doses. An exploratory endpoint was the evaluation of the effect of CRS3123 on fecal microbiome. Thirty healthy male and female subjects, ages 18 to 45, were randomized into three ascending dose cohorts; 200, 400 and 600 mg twice daily for ten days for Cohorts A, B and C, respectively.

Following a single dose of CRS3123, median Tₘₐₓ ranged from 2.00 hours to 3.00 hours across the 3 dose groups and geometric mean t_{1/2} ranged from 3.01 hours to 3.53 hours across the 3 dose groups. Following 10 days of twice daily administration of CRS3123, median T_{max,ss} ranged from 1.00 hour to 2.00 hours across the 3 dose groups and geometric mean t_{1/2,ss} ranged from 4.71 hours to 6.32 hours across the 3 CRS3123 dose groups. There was minimal accumulation of CRS3123 following multiple twice daily dosing for 10 days. Across the dose range studied, geometric mean RAUC ranged from 1.13 to 1.61 and geometric mean RCₘₐₓ ranged from 1.12 to 1.34. This finding is consistent with the relatively short t_{1/2,ss} observed following multiple dosing in relationship to the 12 hour dosing interval. The increase in CRS3123 exposure, based on Cₘₐₓ, and AUC₀₋₁₂ observed on Day 1 and C_{max,ss} and AUC_{0-τ} observed on Day 10, was dose related but was less than proportional to the increase in dose across the dose range studied. Following single dose and multiple dose administration of CRS3123, the majority of unchanged CRS3123 was retained in the GI tract and excreted in feces while renal elimination of CRS3123 as glucuronide conjugates was minimal and accounted for less than 2% of the administered dose.

CRS3123 administered as multiple ascending dose was generally safe and well tolerated with no deaths, other SAEs, or severe TEAEs reported. No subjects were withdrawn from the study due to TEAEs. There were no trends in systemic, vital signs or laboratory TEAEs. The majority of TEAEs reported in the study were of mild severity. There was no prolongation of the QTcF interval or any clinically significant changes in other ECG intervals or morphology.

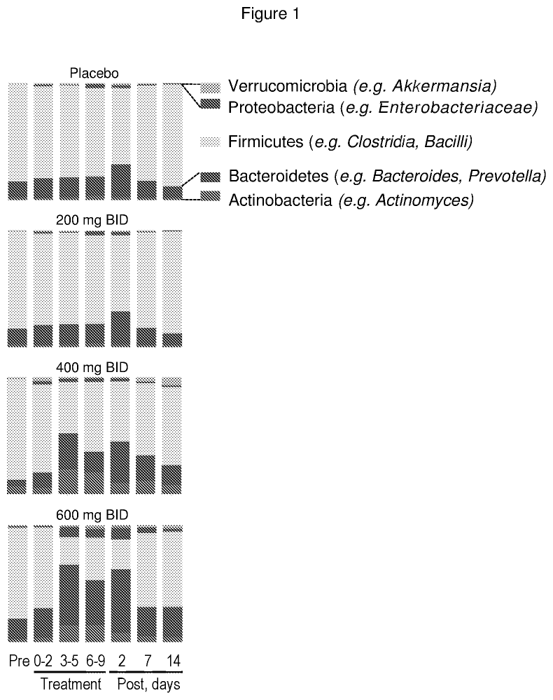

Preliminary microbiome data suggest that most major classes of normal gut flora experienced minimal perturbation during CRS3123 treatment (FIG. 1). The 200 mg BID dose appears to be similar throughout the treatment regimen to placebo. While no phyla are lost at any of the doses, at the two highest doses (400 mg and 600 mg BID), an increase in the proportion of Bacteroidetes is notable, since B. fragilis has been shown to alleviate autism symptoms in MIA mice (Hsiao et al. (2013) supra).

While the invention has been particularly shown and described with reference to a number of embodiments, it would be understood by those skilled in the art that changes in the form and details may be made to the various embodiments disclosed herein without departing from the spirit and scope of the invention and that the various embodiments disclosed herein are not intended to act as limitations on the scope of the claims.

1. A method of treating autism spectrum disorder (ASD) or substantially ameliorating at least one symptom of autism spectrum disorder (ASD) in a mammal comprising administering to the mammal an effective amount of a MetRS inhibitor compound of formula (I), 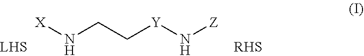
wherein:
X is the left hand side (LHS) substituent and is a substituted or unsubstituted aryl or heteroaryl group;
Z is the right hand side (RHS) substituent and has a substituted or unsubstituted aryl or heteroaryl group; and
Y is a linker group having from one to six methylene groups in a straight chain and in which one or more methylene groups may have one or more (C₁₋₆)alkyl, (C₁₋₆)alkoxy or (C₁₋₆)alkylidenyl substituents.

2. The method of claim 1 wherein the ASD is associated with Clostridium infection.

3. (canceled)

4. The method of claim 1, wherein the MetRS inhibitor is a compound of formula (II): 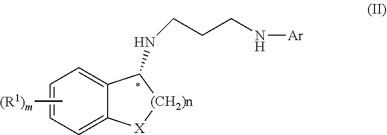
wherein:
Ar is the right hand side (RHS) substituent selected from a substituted or unsubstituted aryl or heteroaryl group;
X is selected from NH, O, S, SO, SO₂, and CH₂;
n is 1, 2 or 3;
m is 0, 1, 2, 3, or 4; and
each R¹ is independently selected from halo, cyano, hydroxyl, (C₁₋₆)alkyl (optionally substituted by halo, hydroxyl, amino, carboxy, or (C₁₋₆)alkoxycarbonyl), (C₃₋₇)cycloalkyl, C(₁₋₆)alkoxy, amino, mono- or di-(C₁₋₆)alkylamino, acylamino, carboxy, (C₁₋₆)alkoxycarbonyl, carboxy(C₁₋₆)alkyloxy, (C₁₋₆)alkylthio, (C₁₋₆)alkylsulphinyl, (C₁₋₆)alkylsulphonyl, sulphamoyl, mono- and di-(C₁₋₆)alkylsulphamoyl, carbamoyl, mono- and di-(C₁₋₆)alkylcarbamoyl and heterocyclyl.

5. (canceled)

6. The method of claim 1 wherein the MetRS inhibitor is:
5-[3-((R)(−)-5,7-Dibromo-1,2,3,4-tetrahydro-naphthalen-1-ylamino)-propylamino]-4H-thieno[3,2-b]pyridine-7-one;
5-[3-((R)(+)-8-Bromo-6-chloro-chroman-4-ylamino)-propylamino]-4H-thieno[3,2-b]pyridine-7-one;
5-[3-((R)(+)-6,8-Dibromo-1,2,3,4-tetrahydro-quinolin-4-ylamino)-propylamino]-4H-thieno[3,2-b]pyridine-7-one;
5-[3-((R)(+)-6,8-Dibromo-chroman-4-ylamino)-propylamino]-4H-thieno[3,2-b]pyridine-7-one;
2-[3-((R)(+)-6,8-Dibromo-chroman-4-ylamino)-propylamino]-1H-quinolin-4-one; or
5-[3-((S)-5,7-Dibromo-benzofuran-3ylamino)-propylamino]-4H-thieno[3,2-b]pyridine-7-one.

7. (canceled)

8. The method of claim 1 wherein the MetRS inhibitor is:
5-[3-(6,8-Dibromo-1,2,3,4-tetrahydro-quinolin-4-ylamino)-propylamino]-4H-thieno[3,2-b]pyridin-7-one;
5-[3-(6,8-Dibromo-chroman-4-ylamino)-propylamino]-4H-thieno[3,2-b]pyridin-7-one;
5-[3-(8-Bromo-6-chloro-chroman-4-ylamino)-propylamino]-4H-thieno[3,2-b]pyridin-7-one;
5-[3-(6-Chloro-8-iodo-chroman-4-ylamino)-propylamino]-4H-thieno[3,2-b]pyridin-7-one;
5-[3-(6-Bromo-8-chloro-chroman-4-ylamino)-propylamino]-4H-thieno[3,2-b]pyridin-7-one;
5-[3-(6-Bromo-8-chloro-1,2,3,4-tetrahydro-quinolin-4-ylamino)-propylamino]-4H-thieno[3,2-b]pyridin-7-one;
5-[3-(8-Bromo-6-chloro-1,2,3,4-tetrahydro-quinolin-4-ylamino)-propylamino]-4H-thieno[3,2-b]pyridin-7-one;
5-[3-(8-Bromo-6-methylsulfanyl-chroman-4-ylamino)-propylamino]-4H-thieno[3,2-b]pyridin-7-one; or
5-[3-(6-Bromo-8-fluoro-1,2,3,4-tetrahydro-quinolin-4-ylamino)-propylamino]-4H-thieno[3,2-b]pyridin-7-one.

9. The method of claim 1 wherein the MetRS inhibitor is a compound of formula (IV): 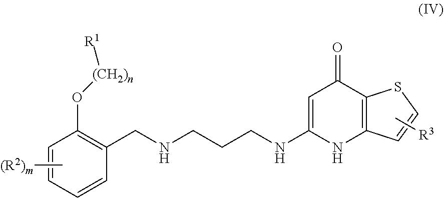
wherein:
R¹ is aryl or heteroaryl;
each R² is independently selected from halo, cyano, hydroxyl, (C₁₋₆)alkyl (optionally substituted by halo, hydroxyl, amino, carboxy, or (C₁₋₆) alkoxycarbonyl), (C₁₋₆)cycloalkyl, (C₁₋₆) alkoxy, amino, mono- or di-(C₁₋₆)alkylamino, acylamino, carboxy, (C₁₋₆)alkoxycarbonyl, carboxy(C₁₋₆)alkyloxy, (C₁₋₆)alkylthio, (C₁₋₆)alkylsulphinyl, (C₁₋₆)alkylsulphonyl, sulphamoyl, mono- and di-(C₁₋₆)alkylsulphamoyl, carbamoyl, and mono- and di-(C₁₋₆)alkylcarbamoyl;
R³ is selected from halo, (C₁₋₃)alkyl, (C₂₋₃)alkenyl, and (C₂₋₃)alkynyl;
n is 1, 2, or 3; and
m is 0, 1, 2 or 3.

10. The method of claim 9, wherein the MetRS inhibitor is:
5-{3-[3-Bromo-5-methylsulfanyl-2-(2-pyridin-3-yl-ethoxy)-benzylamino]-propylamino}-4H-thieno[3,2-b]pyridine-7-one;
5-(3-{3-bromo-5-methylsulfanyl-2-[2-(4-methyl-thiazol-5-yl)-ethoxyl]-benzylamino}-propylamino)-4H-thieno[3,2-b]pyridine-7-one;
5-[3-(3-bromo-5-methylsulfanyl-2-phenethyloxy-benzylamino)-propylamino]-4H-thieno[3,2-b]pyridine-7-one;
5-(3-{3,5-Dibromo-2-[2-(4-methyl-thiazol-5-yl)-ethoxyl]-benzylamino}-propylamino)-4H-thieno[3,2-b]pyridine-7-one;
5-{3-[3,5-Dibromo-2-(2-pyridin-3-yl-ethoxy)-benzylamino]-propylamino}-4H-thieno[3,2-b]pyridine-7-one;
5-(3-f{3,5-Dibromo-2-[2-(4,5-dimethyl-thiazol-2-yl)-ethoxy]-benzylamino}-propylamino)-4H-thieno[3,2-b]pyridine-7-one;
5-[3-(3,5-Dibromo-2-phenethyloxy-benzylamino)-propylamino]-4H-thieno[3,2-b]pyridine-7-one;
5-{3-[3,5-Dibromo-2-(3-pyridin-3-yl-propoxy)-benzylamino]-propylamino}-4H-thieno[3,2-b]pyridine-7-one;
5-(3-{3,5-Dibromo-2-[2-(3,4-dichloro-phenyl)-ethoxy]-benzylamino}-propylamino)-4H-thieno[3,2-b]pyridine-7-one;
5-(3-{3,5-Dibromo-2-[2-(4-methoxy-phenyl)-ethoxy]-benzylamino}-propylamino)-4H-thieno[3,2-b]pyridine-7-one;
5-{3-[3,5-Dibromo-2-(2-p-tolyl-ethoxy)-benzylamino]-propylamino}-4H-thieno[3,2-b]pyridine-7-one;
5-3-f{3,5-Dibromo-2-[2-(fluoro-phenyl)-ethoxy]-benzylamino}-propylamino)-4H-thieno[3,2-b]pyridine-7-one; 5-(3-{3,5-Dibromo-2-[2-(4-chloro-phenyl)-ethoxy]-benzylamino}-propylamino)-4Hthieno[3,2-b]pyridine-7-one; or
5-{3-[3-Bromo-5-methylsulfanyl-2-(3-pyridin-3-yl-propoxy)-benzylamino]-propylamino}-4H-thieno[3,2-b]pyridine-7-one.

11. (canceled)

12. (canceled)

13. (canceled)

14. (canceled)

15. (canceled)

16. (canceled)

17. A method of treating autism spectrum disorder (ASD) or substantially ameliorating at least one symptom of autism spectrum disorder (ASD) in a mammal comprising administering to the mammal an effective amount of a pharmaceutical composition comprising:
a MetRS inhibitor compound of formula (I), 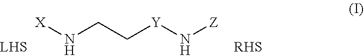
wherein:
X is the left hand side (LHS) substituent and is a substituted or unsubstituted aryl or heteroaryl group;
Z is the right hand side (RHS) substituent and has a substituted or unsubstituted aryl or heteroaryl group; and
Y is a linker group having from one to six methylene groups in a straight chain and in which one or more methylene groups may have one or more (C₁₋₆)alkyl, (C₁₋₆)alkoxy or (C₁₋₆)alkylidenyl substituents; and
a pharmaceutically acceptable carrier or excipient.

18. The method of claim 17, wherein the mammal is a human.

19. The method of claim 18, wherein the ASD is associated with a Clostridium infection.

20. A method of preventing the onset of autism spectrum disorder (ASD) in a mammal comprising administering to the mammal a prophylactically effective amount of a pharmaceutical composition comprising:
a MetRS inhibitor compound of formula (I), 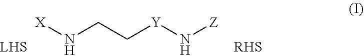
wherein:
X is the left hand side (LHS) substituent and is a substituted or unsubstituted aryl or heteroaryl group;
Z is the right hand side (RHS) substituent and has a substituted or unsubstituted aryl or heteroaryl group; and
Y is a linker group having from one to six methylene groups in a straight chain and in which one or more methylene groups may have one or more (C₁₋₆)alkyl, (C₁₋₆)alkoxy or (C₁₋₆)alkylidenyl substituents; and
a pharmaceutically acceptable carrier or excipient.

21. The method of claim 20, wherein the mammal is a human.

22. The method of claim 21, wherein the ASD is associated with a Clostridium infection.

23. (canceled)

24. (canceled)

25. (canceled)

26. (canceled)

27. (canceled)

28. (canceled)

29. (canceled)

30. The method of claim 17, wherein the microbiota supplementation therapy comprises fecal microbiota transplant.

31. The method of claim 30, wherein the microbiota supplementation therapy comprises at least one of B. fragilis or L. reuteri.

32. (canceled)

33. The method of claim 2, wherein the mammal is a human.
